(19) United States
(12) Patent Application Publication
(10) Pub. No.: US 2026/0117217 A1
(43) Pub. Date: Apr. 30, 2026

(54) ENGINEERED LYSINE DECARBOXYLASES FOR THE PREPARATION OF 1, 5 - DIAMINOPENTANE

(71) Applicant: Enzymaster (Ningbo) Bio-Engineering Co., Ltd., Ningbo, Zhejiang (CN)

(72) Inventors: Baoqin CAI, Ningbo (CN); Xicong DONG; Shijia LIN, Ningbo (CN); Jiyong WANG, Ningbo (CN); Die ZHANG, Ningbo (CN); Marco BOCOLA, Ningbo (CN); Cheng CHEN, Ningbo (CN); Haiben CHEN, Ningbo (CN)

(73) Assignee: Enzymaster (Ningbo) Bio-Engineering Co., Ltd., Ningbo, Zhejiang (CN)

(21) Appl. No.: 19/157,627

(22) PCT Filed: Jan. 30, 2024
(86) PCT No.: PCT/CN2024/074797

§ 371 Date: Aug. 18, 2025

(30) Foreign Application Priority Data
Feb. 18, 2023 (CN) 202310180893.1

(51) Int. Cl. C12N 9/88; C12P 13/00

(52) U.S. Cl. CPC C12N 9/88; C12P 13/001; C12Y 401/01018

(57) ABSTRACT

The present invention provides engineered lysine decarboxylases that can be used to synthesize 1,5-diaminopentane under industrially relevant conditions. The present invention also provides polynucleotides encoding engineered lysine decarboxylases, host cells capable of expressing the engineered lysine decarboxylases, and methods for preparing 1,5-diaminopentane using the engineered lysine decarboxylases. The engineered lysine decarboxylase of the present invention was developed from a wild-type lysine decarboxylase through a creative process of directed evolution, and the engineered lysine decarboxylase of the present invention has a better activity and/or stability and tolerates a high substrate concentration compared to other lysine decarboxylases for the preparation of 1,5-diaminopentane, and thus has a good prospect for industrial application.

### PRIORITY

This application corresponds to the U.S. National phase of International Application No. PCT/CN2024/074797, filed Jan. 30, 2024, which, in turn, claims priority to Chinese Patent Application No. 2023-10180893.1 filed Feb. 18, 2023, the contents of which are incorporated by reference herein in their entirety.

### SEQUENCE LISTING

The instant application contains a Sequence Listing that has been submitted in XML format via EFS-Web and is hereby incorporated by reference in its entirety. Said XML copy, created on Feb. 5, 2024, is named LNK_232US_SEQ.xml and is 389,476 bytes in size.

### TECHNICAL FIELD OF THE PRESENT INVENTION

The present invention relates to the field of bioengineering technology, specifically relates to an engineered lysine decarboxylase and a method for the preparation of 1,5-diaminopentane thereof.

### BACKGROUND OF THE PRESENT INVENTION

1,5-Diaminopentane, also known as cadaverine, is a natural five-carbon nitrogen-containing base with a wide range of applications in agriculture, medicine, and chemicals. In agriculture, 1,5-diaminopentane can participate in a variety of physiological processes in crops, promote androgynous development, and regulate plant aging; in medicine, 1,5-diaminopentane is the precursor for the synthesis of quinolizine, and it can also be utilized as a potent drug for the treatment of dysentery; in the field of chemical engineering, polymer compounds polymerized from 1,5-diaminopentane monomers can be used as chelating and curing agents in a wide range of industrial applications. In addition, the annual global demand for high-performance polyamides has exceeded 6 million tons, and the price is high. In this context, it was found that 1,5-diaminopentane can be polymerized with sebacic acid to form polyamide PA510, which has better performance than polyamides that rely on traditional petrochemical synthesis. Therefore, 1,5-diaminopentane has a highly growing market demand due to its wide applications and excellent properties.

In the prior art, Chong Li et al. from Northwestern University used a chemical method to produce 1,5-diaminopentane by hydrogenation of 1,5-pentanedinitrile as a raw material in the presence of an amorphous nickel catalyst at a pressure of 3 MPa and a temperature of 70° C., and obtained a product with a purity of 66.8%. It can be seen that the chemical synthesis of 1,5-diaminopentane has low selectivity of the product, and the preparation conditions are harsh, costly and polluting; in addition, the chemical method uses non-renewable petroleum as the raw material, and will face the dilemma of petroleum resource scarcity and price increase. Therefore, the development of a bio-enzymatic method for the synthesis of 1,5-diaminopentane is of great significance and will be an inevitable trend under the theme of carbon neutrality.

Currently, the biosynthesis methods of 1,5-diaminopentane mainly include microbial fermentation and whole-cell transformation. The microbial fermentation method usually suffers from the problems of long fermentation period and low yield; in addition, the microbial fermentation system is complicated, with many impurities, which makes it difficult to isolate and purify the products, and thus increases the production cost. Instead, the whole-cell conversion method is different from microbial fermentation in that it enriches the strains through high-density fermentation culture to enhance the enzyme amount, which is then used to catalyze the conversion of the substrate lysine to 1,5-diaminopentane, with the advantage that the system has less impurities and is easy to be purified.

However, the disadvantage of the whole-cell conversion method is that it requires the use of L-lysine as a substrate, with a narrow profit margin between it and the product 1,5-diaminopentane, which leads to the fact that an economically effective enzyme-catalyzed process capable of industrialization would require a high concentration of L-lysine and the ability to fully convert L-lysine to 1,5-diaminopentane, or else it would increase the cost of production and decrease the revenue. Most studies performing whole-cell conversion of 1,5-diaminopentane have been based on the lysine decarboxylase CadA or LdcC of Escherichia coli origin. Patent U.S. Pat. No. 7,189,543B2 discloses a method to produce 1,5-diaminopentane by adjusting pH with dicarboxylic acid and converting lysine by cellular overexpression of wild-type lysine decarboxylase polypeptide; however, the disadvantages of this method are that the substrate concentration is too low at only 50 g/L, and the ratio of the substrate feed to the enzyme dosage (substrate to enzyme ratio) is less than 7.5. It has been reported to use the Escherichia coli XL1-Blue engineered bacterium overexpressing LdcC, the initial bacterial OD₆₀₀=50, the initial concentration of lysine rose to 200 g/L. However, the disadvantage is that it takes 120 hours of catalytic reaction to obtain 133.75 g/L of 1,5-diaminopentane product, the reaction time is long, and the concentration of the product is still low. Patent CN107164352B disclosed a number of CadA mutants, in which 0.2 g/L dry weight of the engineering bacteria (M176V/Y230H/K44R) achieved 84% conversion of 25 g/L substrate after 4 hours at 37° C. The disadvantage of this method is still that the concentration of the product is too low, and therefore does not have the advantage of industrialization of scale-up production. Patents CN102851307A and CN104762336A disclosed engineered lysine decarboxylase derived from the bacterium Hafnia alvei, which was used to catalyze the decarboxylation of L-lysine for the production of 1,5-diaminopentane; in which the feeding load of the substrate lysine hydrochloride rose to 450 g/kg, the substrate to enzyme ratio was about 10, and the catalytic reaction yielded 251 g/kg of 1,5-diaminopentane after 48 hours of catalytic reaction, which has been greatly improved in terms of catalytic efficiency and temporal and spatial yields overall. The disadvantage is that the activity of lysine decarboxylase is not enough to cause the catalytic reaction time is too long, which is inefficient and leads to high production cost.

### SUMMARY OF THE PRESENT INVENTION

The present invention provides an engineered lysine decarboxylase polypeptide. Under the condition of substrate L-lysine hydrochloride feeding load up to 650 g/L, only a small amount of wet cells expressing the engineered lysine decarboxylase polypeptide (substrate enzyme ratio of 100:1) was used, and the substrate could be completely converted to 1,5-diaminopentane after a catalytic reaction of only 6 hours. The present invention also provides the gene and protein sequence of the engineered lysine decarboxylase polypeptide, a recombinant expression vector containing the gene, an engineered bacterial strain and an efficient method for its preparation, and a reaction process for the preparation of 1,5-diaminopentane using the engineered polypeptide.

A first aspect of the present invention provides an improved engineered lysine decarboxylase polypeptide. This engineered polypeptide is derived from a wild-type lysine decarboxylase that has been modified by an artificially directed evolutionary process with a certain number of mutations such as amino acid residue substitutions, insertions or deletions. The wild-type lysine decarboxylase is derived from Plesiomonas shigelloides and its sequence is shown in SEQ ID No: 2. This wild type lysine decarboxylase has low activity, poor thermal stability, and product inhibition towards 1.5-diaminopentane. As tested by the inventors, when using the lysine decarboxylase of SEQ ID No: 2 for the reaction shown below, no more 1.5-diaminopentane is generated after 2 hours of the reaction (reaction temperature of 35° C.-40° C.) at a loading level of 450 g/L of L-lysine hydrochloride, and the conversion rate does not exceed 20%.

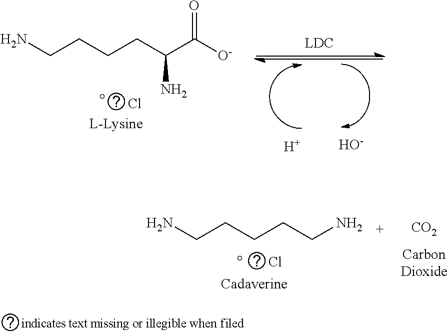

The present invention provides improved engineered lysine decarboxylase polypeptides that have higher activity and/or stability, overcome product inhibition, and are able to catalyze the generation of 1.5-diaminopentane from L-lysine more efficiently compared to the wild-type lysine decarboxylase corresponding to SEQ ID NO: 2. These improved lysine decarboxylase polypeptides may comprise an amino acid sequence that differs from the SEQ ID NO: 2 sequence by one or more residues at residue positions corresponding to the following: X5, X11, X12, X13, X24, X48, X85, X95, X99, X108, X111, X116, X119, X143, X144, X145, X316, X334, X368, X378, X383, X422, X440, X441, X445, X516, X521, X551, X561, X591, X595, X693, X694, X701, X710. Improved lysine decarboxylase polypeptides comprising an amino acid sequence comprising at least one of the following features: A5V, G11A, G11C, G11P, G11R, G11T, G11S, A12E, A12V, A12R, A12L, A12T, A12Q, A12K, F131, F13W, F13V, F13P, F13Y, A24K, N48S, T85D, D95G, N99D, N99A, N99G, N99Y, N99T, N99S, N99Q, D108Q, S111I, K116Q, Q119P, Q119D, Q119R, G143P, K144L, K144F, K144Q, K144R, K144E, Y145D, Y145V, Y145W, Y145L, D316E, V3341, L368C, 1378V, D383E, K422Q, K440R, K441Q, K441R, E445Y, E445L, E445A, K516R, R521L, R521M, R551L, R551Q, R551N, D561E, Q591G, Q591S, K595R, 1693V, H694Q, D701G, K710Q, or both on the basis of these differences, comprising an insertion or deletion of 1, 2, 3, 4, 5, or more amino acid residues.

More specifically, in some embodiments, an engineered lysine decarboxylase polypeptide improved upon SEQ ID NO: 2 comprises a polypeptide comprising the amino acid sequence corresponding to SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146,148,150,152,154,156,158,160,162,164,166,168,170.

In some embodiments, an improved engineered lysine decarboxylase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to the reference sequence of SEQ ID No: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148,150,152,154,156,158,160,162,164,166,168,170.

The identity between two amino acid sequences or two nucleotide sequences can be obtained by algorithms commonly used in the art, which can be calculated using NCBI Blastp and Blastn software based on default parameters, or by employing the Clustal W algorithm (Nucleic Acid Research, 22(22):4673-4680, 1994). For example, with using the Clustal W algorithm, the amino acid sequence identity between SEQID NO: 2 and SEQ ID NO: 68 is 99.3%.

In another aspect, the present invention provides polynucleotide sequences encoding engineered lysine decarboxylase polypeptides. In some embodiments, the polynucleotide may be a portion of an expression vector having one or more control sequences for expressing the engineered lysine decarboxylase polypeptide. In some embodiments, the polynucleotide may comprise a polynucleotide sequence corresponding to SEQ ID No: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169.

As is known to those of skill in the art, due to the degeneracy of the nucleotide codons, the polynucleotide sequences encoding the amino acid sequences of SEQ ID No: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170 are not limited to SEQ ID No: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169. The nucleic acid sequence encoding the lysine decarboxylase of the present invention may also be any other nucleic acid sequence encoding the amino acid sequence shown in SEQ ID No: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170.

In another aspect, the present disclosure provides expression vectors and host cells comprising a polynucleotide encoding an engineered lysine decarboxylase or capable of expressing an engineered lysine decarboxylase. In some embodiments, the host cell may be a bacterial host cell, such as E. coli. The host cell may be used to express and isolate an engineered lysine decarboxylase as described herein, or optionally used directly to reactively transform the substrate into a product.

In some embodiments, engineered lysine decarboxylase in the form of intact cells, crude extracts, isolated polypeptides, or purified polypeptides may be used alone, or in an immobilized form (e.g., immobilized on a resin).

The present disclosure also provides methods of converting an L-lysine compound shown in structural formula (II)

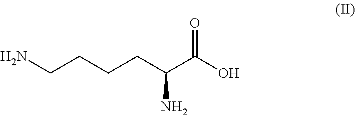

to a 1,5-diaminopentane compound shown in structural formula (I) using an engineered lysine decarboxylase polypeptide disclosed herein.

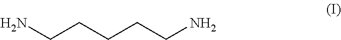

Specific embodiments of engineered lysine decarboxylase polypeptides for use in the methods are provided further in the Detailed Description. Improved engineered lysine decarboxylase polypeptides usable in the methods described above may comprise a polypeptide having amino acid sequences selected from the group corresponding to SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170.

Any of the methods of using an engineered peptide for the preparation of a compound of formula (I) as disclosed herein may be performed under a range of suitable reaction conditions, said range of suitable reaction conditions including, but not limited to, a range of pH, temperature, substrate loading, peptide loading, cofactor loading, and reaction time. For example, in some embodiments, preparation of a compound of Formula (I) may be performed wherein suitable reaction conditions include (a) from about 10 g/L to about 650 g/L of substrate compound (II); (b) from about 0.1 g/L to about 50 g/L of engineered polypeptide; (c) a PLP cofactor concentration of from about 0.1 mM to about 5 mM; (d) a pH of from about 5.5 to about 8.5; (e) a temperature of from about 10° C. to 60° C.; and (f) a reaction time of about 1 hour to about 12 hours.

### DETAILED DESCRIPTION OF THE PREFERRED EMBODIMENTS

### Improved Engineered Lysine Decarboxylase

The engineered lysine decarboxylase polypeptides developed by the present invention are exemplified in Table 1 below. Each row gives the polynucleotide sequence number and amino acid sequence number of a specific engineered lysine decarboxylase polypeptide, as well as residue differences compared to SEQ ID No:2. The level of catalytic performance (overall performance in the reaction, combining activity, stability and overcoming product inhibition) of each exemplified engineered lysine decarboxylase polypeptide is indicated by a “+”, the specific meaning of which is shown in Table 2.

TABLE 1
Engineered lysine decarboxylase polypeptides

| | | | Sequence | |
|---|---|---|---|---|
| | | | identity | |
| polynucleotide | Amino acid | | with SEQ | Catalytic |
| sequence | sequence | Residue differences relative to SEQ ID No: 2 | ID No: 2 | performance |
| 1 | 2 | — | 100% | |
| 3 | 4 | S111I; | 99.8% | + |
| 5 | 6 | R551L; | 99.8% | + |
| 7 | 8 | N99D; | 99.8% | + |
| 9 | 10 | K144L; | 99.8% | + |
| 11 | 12 | D108Q; | 99.8% | + |
| 13 | 14 | R551Q; | 99.8% | + |
| 15 | 16 | E445Y; | 99.8% | + |
| 17 | 18 | K144F; | 99.8% | + |
| 19 | 20 | F13I; | 99.8% | ++ |
| 21 | 22 | A12E; | 99.8% | ++ |
| 23 | 24 | K144Q; | 99.8% | ++ |
| 25 | 26 | A5V; N48S; | 99.7% | ++ |
| 27 | 28 | D95G; D701G; | 99.7% | ++ |
| 29 | 30 | K144Q; K595R; | 99.7% | ++ |
| 31 | 32 | K144Q; H694Q; | 99.7% | ++ |
| 33 | 34 | K144Q; K441Q; | 99.7% | ++ |
| 35 | 36 | K144Q; K441R; K516R; D561E; K595R; | 99.3% | ++ |
| 37 | 38 | A12V; K144Q; K595R; | 99.6% | ++ |
| 39 | 40 | K144Q; E445L; K595R; | 99.6% | ++ |
| 41 | 42 | T85D; Q144R; K595R; | 99.6% | ++ |
| 43 | 44 | K144Q; K441Q; R551N; K595R; | 99.4% | ++ |
| 45 | 46 | A24K; K441Q; R551N; K595R; | 99.4% | ++ |
| 47 | 48 | K116Q; K144Q; K595R; | 99.6% | ++ |
| 49 | 50 | Q144E; K595R; | 99.6% | ++ |
| 51 | 52 | N99A; K144Q; K595R; | 99.6% | ++ |
| 53 | 54 | K144Q; E445A; K595R; | 99.6% | ++ |
| 55 | 56 | G11A; K144Q; K595R; | 99.6% | ++ |
| 57 | 58 | K144Q; D561E; | 99.7% | ++ |
| 59 | 60 | K144Q; K440R; K516R; K595R; | 99.4% | ++ |
| 61 | 62 | N99G; K144Q; K595R; | 99.6% | ++ |
| 63 | 64 | N99Y; K144Q; R551N; | 99.6% | ++ |
| 65 | 66 | K144Q; K440R; K441R; K516R; K595R; | 99.3% | ++ |
| 67 | 68 | K144Q; K441R; K516R; K595R; K710Q; | 99.3% | ++ |
| 69 | 70 | A24K; K144Q; R551N; K595R; | 99.4% | ++ |
| 71 | 72 | K144Q; K441Q; K595R; K710Q; | 99.4% | ++ |
| 73 | 74 | G11C; K144Q; K595R; | 99.6% | ++ |
| 75 | 76 | K144Q; K516R; K595R; K710Q; | 99.4% | ++ |
| 77 | 78 | K144Q; K516R; | 99.7% | ++ |
| 79 | 80 | K144Q; D383E; | 99.7% | ++ |
| 81 | 82 | K144Q; K441R; K516R; K595R; | 99.4% | ++ |
| 83 | 84 | K144Q; K516R; K595R; | 99.6% | +++ |
| 85 | 86 | K144Q; K440R; K595R; K710Q; | 99.4% | +++ |
| 87 | 88 | K144Q; I378V; K595R; | 99.6% | +++ |
| 89 | 90 | K144Q; V334I; K595R; | 99.6% | +++ |
| 91 | 92 | D383E; K144Q; K516R; K595R; | 99.4% | +++ |
| 93 | 94 | K144Q; K440R; K595R; | 99.6% | +++ |
| 95 | 96 | A24K; N99Y; K144Q; | 99.6% | +++ |
| 97 | 98 | N99T; K144Q; K595R; | 99.6% | +++ |
| 99 | 100 | K144Q; K422Q; | 99.7% | +++ |
| 101 | 102 | N99S; K144Q; K595R; | 99.6% | +++ |
| 103 | 104 | K144Q; K440R; K441Q; K595R; | 99.4% | +++ |
| 105 | 106 | K144Q; D316E; K440R; K595R; | 99.4% | +++ |
| 107 | 108 | K144Q; L368C; K595R; | 99.6% | +++ |
| 109 | 110 | K144Q; Q591G; K595R; | 99.6% | +++ |
| 111 | 112 | K144Q; Q591S; K595R; | 99.6% | +++ |
| 113 | 114 | N99Q; K144Q; K595R; | 99.6% | +++ |
| 115 | 116 | K144Q; K595R; I693V; | 99.6% | +++ |
| 117 | 118 | K144Q; Y145D; K595R; | 99.6% | +++ |
| 119 | 120 | K144Q; D316E; | 99.7% | +++ |
| 121 | 122 | K144Q; K595R; H694Q; | 99.6% | +++ |
| 123 | 124 | Q119P; K144Q; K595R; | 99.6% | +++ |
| 125 | 126 | K144Q; Y145V; K595R; | 99.6% | +++ |
| 127 | 128 | A12E; K144Q; K595R; | 99.6% | +++ |
| 129 | 130 | A12R; K144Q; K595R; | 99.6% | +++ |
| 131 | 132 | F13W; K144Q; K595R; | 99.6% | +++ |
| 133 | 134 | A12L; K144Q; K595R; | 99.6% | +++ |
| 135 | 136 | F13V; K144Q; K595R; | 99.6% | +++ |
| 137 | 138 | G11P; K144Q; K595R; | 99.6% | ++++ |
| 139 | 140 | K144Q; Y145W; K595R; | 99.6% | ++++ |
| 141 | 142 | G143P; K144Q; K595R; | 99.6% | ++++ |
| 143 | 144 | F13P; K144Q; K595R; | 99.6% | ++++ |
| 145 | 146 | A12T; K144Q; K595R; | 99.6% | ++++ |
| 147 | 148 | F13Y; K144Q; K595R; | 99.6% | ++++ |
| 149 | 150 | G11R; K144Q; K595R; | 99.6% | ++++ |
| 151 | 152 | K144Q; R521L; K595R; | 99.6% | ++++ |
| 153 | 154 | F13I; K144Q; K595R; | 99.6% | ++++ |
| 155 | 156 | Q119D; K144Q; K595R; | 99.6% | ++++ |
| 157 | 158 | G11T; K144Q; K595R; | 99.6% | ++++ |
| 159 | 160 | K144Q; Y145L; K595R; | 99.6% | ++++ |
| 161 | 162 | K144Q; R521M; K595R; | 99.6% | ++++ |
| 163 | 164 | A12Q; K144Q; K595R; | 99.6% | ++++ |
| 165 | 166 | A12K; K144Q; K595R; | 99.6% | +++++ |
| 167 | 168 | G11S; K144Q; K595R; | 99.6% | +++++ |
| 169 | 170 | Q119R; K144Q; K595R; | 99.6% | +++++ |

TABLE 2
Catalytic properties and assay conditions of engineered lysine decarboxylase polypeptides

| | Increased catalytic properties of | |
|---|---|---|
| Representative | engineered lysine decarboxylase | |
| symbols | polypeptides | Measurement conditions |
| + | 1.2-2.5 times of the conversion | L-lysine hydrochloride 450 g/L, L-lysine |
| | rate of SEQ ID No: 2 | hydrochloride 10 g/L, PLP 0.5 mM, 35° C. |
| ++ | 2.5-5 times of the conversion | L-lysine hydrochloride 450 g/L, L-lysine |
| | rate of SEQ ID No: 2 | hydrochloride 4.5 g/L, PLP 0.25 mM, 35° C. |
| +++ | 5-10 times of the conversion | L-lysine hydrochloride 450 g/L, L-lysine |
| | rate of SEQ ID No: 2 | hydrochloride 4.5 g/L, PLP 0.5 mM, 45° C. |
| ++++ | 10-20 times of the conversion | L-lysine hydrochloride 450 g/L, L-lysine |
| | rate of SEQ ID No: 2 | hydrochloride 4.5 g/L, PLP 0.1 mM, 45° C. |
| +++++ | 20-25 times of the conversion | L-lysine hydrochloride 450 g/L, L-lysine |
| | rate of SEQ ID No: 2 | hydrochloride 4.5 g/L, PLP 0.1 mM, 45° C. |

The amino acid sequences listed in Table 1 (i.e., even sequence identifiers of SEQ ID NO:2-170) all contain 711 amino acid residues. The wet cells, described in Table 2, contains nearly equal amounts of engineered lysine decarboxylase polypeptide protein expression.

The wild-type enzyme SEQ ID NO:2 is less active, less thermally stable, and subject to product inhibition. The improved engineered lysine decarboxylase polypeptide provided by the present invention has higher activity, higher stability, overcomes product inhibition, and catalyzes a higher conversion of L-lysine to 1.5-diaminopentane compared to the wild-type lysine decarboxylase corresponding to SEQ ID NO: 2.

Polynucleotides, Control Sequences, Expression Vectors and Host Cells that can be Used to Produce Engineered Lysine Decarboxylase Polypeptides

In another aspect, the present disclosure provides polynucleotides encoding engineered polypeptides having lysine decarboxylase activity as described herein. The polynucleotides may be operably linked to one or more heterologous regulatory sequences that control gene expression to produce a recombinant polynucleotide that are capable of expressing the polypeptide. Expression constructs comprising heterologous polynucleotides encoding engineered lysine decarboxylase may be introduced into suitable host cells to express the corresponding engineered lysine decarboxylase polypeptide.

As apparent to those of skill in the art, the availability of protein sequences and knowledge of codons corresponding to a variety of amino acids provide an illustration of all possible polynucleotides that encode a target protein sequence. The degeneracy of the genetic code, in which the same amino acids are encoded by selectable or synonymous codons, allows for the production of an extremely large number of polynucleotides, all of which encode the improved lysine decarboxylase polypeptides disclosed herein. Thus, upon determination of a particular amino acid sequence, one of skill in the art can generate any number of different polynucleotides by merely modifying the sequence of one or more codons in a manner that does not alter the amino acid sequence of the protein. In this regard, the present disclosure particularly contemplates each and every possible alteration of polynucleotides that can be made by selecting a combination based on possible codon selections, and any of the polypeptides disclosed herein, comprising the amino acid sequences of the exemplary engineered polypeptides provided in Table 1, and in the sequence lists incorporated herein by reference as an even numbered sequences of SEQ IDNO:4-170 Identifier of the sequence of any polypeptide disclosed, all such changes are considered specifically disclosed.

In a variety of embodiments, the codons are preferably selected to accommodate the host cell in which the recombinant protein is produced. For example, codons preferred for bacteria are used to express genes in bacteria; codons preferred for yeast are used to express genes in yeast; and codons preferred for mammals are used for gene expression in mammalian cells.

In some embodiments, the polynucleotides encode Lysine decarboxylase polypeptides comprising amino acid sequences that are at least about 80%, 85%, 86%, 87%, 88%, 89%, 90%, 91%, 92%, 93%, 94%, 95%, 96%, 97%, 98%, 99%, or more sequence identical to a reference sequence that is an even sequence identifiers of SEQ ID NO: 4-170, wherein the polypeptides have lysine decarboxylase activity and one or more of the improved properties described herein, such as the ability to convert lysine to the product 1,5-diaminopentane with increased activity compared to the polypeptide of SEQ ID NO:2.

In some embodiments, the polynucleotide encodes an engineered lysine decarboxylase polypeptide, the engineered lysine decarboxylase polypeptide comprising an amino acid sequence that having a percentage of identity as described above as compared to SEQ ID NO:2 and having one or more amino acid residue differences selected. In some embodiments, the present disclosure provides engineered polypeptides having lysine decarboxylase activity, the engineered polypeptide comprises a combination of a reference sequence having at least 99.3% sequence identity to the reference sequence of SEQ ID NO:2 and a residue difference at a position selected from: X5, X11, X12, X13, X24, X48, X85, X95, X99, X108, X111, X116, X119, X143, X144, X145, X316, X334, X368, X378, X383, X422, X440, X441, X445, X516, X521, X551, X561, X591, X595, X693, X694, X701, X710.

In some embodiments, the polynucleotides encoding the engineered lysine decarboxylase polypeptides comprise sequences having odd sequence identifiers of SEQ ID NO:3-169.

In some embodiments, the polynucleotides encode polypeptides as described herein, but at the nucleotide level, the polynucleotide has about 50%, 60%, 70%, 80%, 85%, 90%, 91%, 92%, 93%, 94%, 95%, 96%, 97%, 98%, or 99% or more sequence identity to reference polynucleotides encoding engineered lysine decarboxylase. In some embodiments, the reference polynucleotide sequence is selected from a sequence having the odd sequence identifier of SEQ ID NO:3-169.

The isolated polynucleotides encoding the engineered lysine decarboxylase polypeptide can be manipulated to enable the expression of the polypeptides in a variety of ways, which comprises further modification of the sequence by codon optimization to improve expression, insertion into suitable expression elements with or without additional control sequences, and transformation into host cells suitable for expression and production of the engineered polypeptide.

Depending on the expression vector, manipulation of the isolated polynucleotide prior to insertion of the isolated polynucleotide into the vector may be desirable or necessary. Techniques for modifying polynucleotides and nucleic acid sequences using recombinant DNA methods are well known in the art. Guidance is provided below: Sambrook et al, 2001, MolecuLar Cloning: A Laboratory Manual, Third Edition, Cold Spring Harbor Laboratory Press; and Current Protocols in MolecuLar Biology, edited by Ausubel. F., GreenePub.Associates, 1998, updated 2010.

In another aspect, the present disclosure also relates to recombinant expression vectors, depending on the type of host they are to be introduced into, including a polynucleotide encoding an engineered lysine decarboxylase polypeptide or variant thereof, and one or more expression regulatory regions, such as promoters and terminators, origin of replication and the like. Alternatively, the nucleic acid sequences of the present disclosure can be expressed by inserting the nucleic acid sequence or the nucleic acid construct comprising the sequence into an appropriate expression vector. In generating an expression vector, the coding sequence is located in the vector such that the coding sequence is linked to a suitable control sequence for expression.

The recombinant expression vector may be any vector (e.g., a plasmid or a virus) that can be conveniently used in the recombinant DNA procedures and can result in the expression of a polynucleotide sequence. The choice of vector will generally depend on the compatibility of the vector with the host cell to be introduced into. The vector may be a linear or closed circular plasmid. The expression vector may be an autonomously replicating vector, i.e., a vector that exists as an extrachromosomal entity whose replication is independent of chromosomal replication, such as a plasmid, extrachromosomal element, minichromosome, or artificial chromosomes. The vector may contain any tools for ensuring self-replication. Alternatively, the vector may be a vector that, when introduced into a host cell, integrates into the genome and replicates with the chromosome into which it is integrated. Moreover, a single vector or plasmid or two or more vectors or plasmids that together comprise the total DNA to be introduced into the genome of the host cell may be used.

Many expression vectors useful to the embodiments of the present disclosure are commercially available. Exemplary expression vectors can be prepared by inserting a polynucleotide encoding an improved lysine decarboxylase polypeptide to the plasmid pACYC-Duet-1 (Novagen).

In another aspect, the present disclosure provides host cells comprising polynucleotides encoding engineered lysine decarboxylase polypeptides of the present disclosure, the polynucleotide is linked to one or more control sequences for expression of lysine decarboxylase in the host cell. Host cells for expression of polypeptides encoded by expression vectors of the present disclosure are well known in the art, including, but are not limited to, bacterial cells such as Escherichia coli, Arthrobacter spp. KNK168, Streptomyces, and Salmonella typhimurium cells; fungal cells such as yeast cells (e.g., Saccharomyces cerevisiae or Pichia pastoris); insect cells such as Drosophila S2 and Spodoptera Sf9 cells; animal cells such as CHO, COS, BHK, 293 and Bowes melanoma cells; and plant cells. An exemplary host cell is E. coli BL21 (DE3). The above host cells may be wild-type or engineered cells through genomic edition, such as knockout of the wild-type lysine decarboxylase gene carried in the host cell's genome. Suitable media and growth conditions for the above host cells are well known in the art.

Polynucleotides used to express engineered lysine decarboxylase can be introduced into the cells by a variety of methods known in the art. Techniques comprise, among others, electroporation, bio-particle bombardment, liposome-mediated transfection, calcium chloride transfection, and protoplast fusion. Different methods of introducing polynucleotides into cells are obvious to those skilled in the art.

### Process of Preparing Engineered Lysine Decarboxylase Polypeptides

Engineered lysine decarboxylase can be obtained by subjecting a polynucleotide encoding lysine decarboxylase to mutagenesis and/or directed evolution methods. Exemplary directed evolution techniques can be found in Biocatalysis for the Pharmaceutical Industry:Discovery, Development, and Manufacturing (2009 John Wiley&Sons Asia (Pte) Ltd. ISBN. 978-0-470-82314-9).

When the sequence of the engineered polypeptide is known, the encoding polynucleotide may be prepared by standard solid-phase methods according to known synthetic methods. In some embodiments, fragments of up to about 100 bases can be synthesized separately and then ligated (e.g., by enzymatic or chemical ligation methods or polymerase-mediated methods) to form any desired contiguous sequence. For example, the polynucleotides and oligonucleotides of the present disclosure can be prepared by chemical synthesis using, for example, the classical phosphoramidite method described by Beaucage et al, 1981, TetLett 22:1859-69, or the method described by Matthes et al,1984, EMBOJ.3:801-05, as typically practiced in automated synthetic methods. According to the phosphoramidite method, oligonucleotides are synthesized, purified, annealed, ligated, and cloned into a suitable vector, for example, in an automated DNA synthesizer. In addition, essentially any nucleic acid is available from any of a variety of commercial sources.

In some embodiments, the present disclosure also provides a process for preparing or producing an engineered lysine decarboxylase polypeptide, wherein the process comprises culturing a host cell capable of expressing a polynucleotide encoding the engineered polypeptide under culture conditions suitable for expression of the polypeptide. In some embodiments, the process of preparing a polypeptide further comprises isolating the polypeptide. The engineered polypeptide may be expressed in a suitable cell and isolated (or recovered) from the host cell and/or culture medium using any one or more of the techniques known for protein purification, the techniques for protein purification include, among others, lysozyme treatment, sonication, filtration, salting out, ultracentrifugation and chromatography.

### Methods of Utilizing Engineered Lysine Decarboxylase and Compounds Prepared Therewith

In another aspect, the improved engineered lysine decarboxylase polypeptides described herein convert L-lysine to 1,5-diaminopentane. In some embodiments, the engineered lysine decarboxylase polypeptide can be used in a process of preparing a compound of structural formula (I),

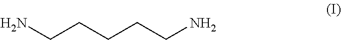

wherein, including the step of contacting L-lysine with an improved engineered lysine decarboxylase polypeptide disclosed herein.

In some embodiments of the above process, the compound of formula (I) is produced at a conversion of at least 70%, 75%, 80%, 85%, 90%, 91%, 92%, 93%, 94%, 95%, 96%, 97%, 98%, 99% or more.

Specific embodiments of engineered lysine decarboxylase polypeptides for use in the above process are further provided in the detailed description. Engineered lysine decarboxylase polypeptides that can be used in the above process comprise amino acid sequences selected from SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150,152,154,156,158,160,162,164,166,168,170.

As described herein and exemplified in the Examples, the present disclosure contemplates a range of suitable reaction conditions that may be used in the process herein, including but not limited to, pH, temperature, substrate loading, polypeptide loading, and reaction time. Additional suitable reaction conditions for performing a method of biocatalytically converting substrate compounds to product compounds using engineered lysine decarboxylase polypeptides described herein can be readily optimized by routine experimentation, which includes but is not limited to, the engineered lysine decarboxylase polypeptide is contacted with the substrate compound under experimental reaction conditions of varying the loading of individual reaction components, pH, and temperature conditions, and the product compound is detected. For example, utilizing the methods described in the embodiments provided herein.

As described above, engineered polypeptides having lysine decarboxylase activity for use in the process of the present disclosure generally comprise amino acid sequences that have at least 90%, 91%, 92%, 93%, 94%, 95%, 96%, 97%, 98%, 99% or more sequence identity to the reference amino acid sequence selected from any one of the even numbered sequences of SEQ ID NO:4-170.

The loading of substrate compounds in the reaction mixture can be varied, taking into consideration of, for example, the amount of the desired product compound, the effect of the substrate concentration on the enzyme activity, the stability of the enzyme under the reaction conditions, and the conversion of substrate to product. In some embodiments of the process, the suitable reaction conditions include at least about 10 g/L, at least about 15 g/L, at least about 20 g/L, at least about 30 g/L, at least about 50 g/L, at least about 75 g/L, at least about 100 g/L, at least about 150 g/L, at least about 200 g/L, at least about 250 g/L, at least about 300 g/L, at least about 350 g/L, at least about 400 g/L, at least about 450 g/L, at least about 500 g/L, and at least about 650 g/L of a substrate L-lysine hydrochloride loading. The values for the substrate loading provided herein are based on the molecular weight, however it is also contemplated that the equivalent molar amounts of various hydrates and salts of compound (II) may also be used in the process.

In the processes described herein, an engineered lysine decarboxylase polypeptide catalyzes the decarboxylation of L-lysine to form 1.5-diaminopentane. In some embodiments, the lysine in the reaction is L-lysine, including the option to be applied in the form of a salt (e.g., lysine hydrochloride, lysine sulfate, etc.) in the embodiment.

In some embodiments, suitable reaction conditions include a solution pH of about 5.5 to about 8.5. In some embodiments, the reaction conditions include a solution pH of about 5, 5.5, 6, 6.5, 7, 7.5, 8, 8.5.

In embodiments of the processes herein, suitable temperatures may be used for reaction conditions, taking into consideration of, for example, the increase in reaction rate at higher temperatures, and the activity of the enzyme for sufficient duration of reaction. Accordingly, in some embodiments, suitable reaction conditions include a temperature of about 10° C. to about 60° C., about 25° C. to about 50° C., about 25° C. to about 45° C., or about 30° C. to about 40° C. In some embodiments, suitable reaction temperatures include a temperature of about 25° C., 30° C., 35° C., 40° C., or 45° C. In some embodiments, the temperature during the enzymatic reaction may be maintained at a certain temperature throughout the reaction.

The process of the use of engineered lysine decarboxylase is usually carried out in water or a solvent. The carbon dioxide produced during the decarboxylation reaction has the potential to cause the formation of foams, and an antifoam agent may be added as appropriate.

Suitable reaction conditions may include combinations of reaction parameters that allows the biocatalytic conversion of the substrate compound to its corresponding product compound. Accordingly, in some embodiments of the process, the combination of reaction parameters comprises: (a) a loading of the substrate L-lysine hydrochloride of from about 10 g/L to about 650 g/L; (b) a loading of the engineered polypeptide of from about 0.1 g/L to about 10 g/L; (c) a concentration of the PLP cofactor of from about 0.1 mM to about 5 mM; (d) a pH of from about 5.5 to about 8.5; (e) a temperature of from about 10° C. to about 60° C.; and (f) a time from about 1 hour to about 24 hours.

In some embodiments, the method described above comprises contacting 10 g/L of L-lysine hydrochloride substrate with 0.1 g/L of the engineered lysine decarboxylase polypeptide wet cells described herein at a temperature of from about 10° C. to about 60° C. and a pH of from about 5.5 to about 8.5 for a reaction duration from about 1 h to about 24 h, at least 70%, 80%, 90%, or more of the L-lysine hydrochloride substrate is converted to 1,5-diaminopentane product. In some embodiments, the lysine decarboxylase polypeptide capable of performing the above-described reaction comprises the amino acid sequence selected from SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170.

Exemplary reaction conditions include the assay conditions provided in Table 2 and Examples 7 and 8.

In carrying out the decarboxylation reactions described herein, the engineered lysine decarboxylase polypeptide may be added to the reaction mixture in the partially purified or purified forms, whole cells transformed with the gene encoding the engineered lysine decarboxylase polypeptide, and/or as cell extracts and/or lysates of such cells. Whole cells transformed with the gene encoding the engineered lysine decarboxylase or cell extracts, lysates thereof, and isolated enzymes can be used in a wide variety of different forms, including solids (e.g., lyophilized, spray dried, or the like) or semi-solid (e.g., a crude paste such as wet cells). The cell extract or cell lysate may be partially purified by precipitation (e.g., ammonium sulfate, polyethyleneimine, heat treatment or the like), followed by desalting procedures (e.g., ultrafiltration, dialysis, and the like) prior to lyophilization. Any of the enzyme preparations can be stabilized by crosslinking using known crosslinking agents, such as glutaraldehyde, or immobilization to a solid phase material (such as a resin).

In some embodiments of the decarboxylation reaction described herein, the reaction is carried out under suitable reaction conditions described herein, wherein the engineered lysine decarboxylase polypeptide is immobilized onto a solid support. Solid supports useful for immobilizing the engineered lysine decarboxylase enzyme for carrying out the reaction include but are not limited to beads or resins such as polymethacrylates with epoxy functional groups, polymethacrylates with amino epoxy functional groups, polymethacrylates, styrene/DVB copolymer or polymethacrylates with octadecyl functional groups. Exemplary solid supports include, but are not limited to, chitosan beads, Eupergit C, and SEPABEADs (Mitsubishi), including the following different types of SEPABEAD: EC-EP, EC-HFA/S, EXA252, EXE119 and EXE120.

In some embodiments, wherein the engineered polypeptide may be expressed in the form of a secreted polypeptide, a culture medium containing the secreted polypeptide can be used in the process herein.

In some embodiments, the solid reactants (e.g., enzymes, salts, etc.) may be provided to the reaction in a variety of different forms, including powders (e.g., lyophilized, spray dried, etc.), solutions, emulsions, suspensions and the like. The reactants can be readily lyophilized or spray-dried using methods and instrumentation known to one skilled in the art. For example, the protein solution can be frozen at −80° C. in small aliquots, and then added to the pre-chilled lyophilization chamber, followed by the application of a vacuum.

In some embodiments, the order of addition of reactants is not critical. The reactants may be added together to the solvent at the same time (e.g., monophasic solvent, a biphasic aqueous co-solvent system, etc.), or alternatively, some reactants may be added separately, and some may be added together at different time points.

### EXAMPLES

The following examples further illustrate the present invention, but the present invention is not limited thereto. In the following examples, experimental methods with conditions not specified, were conducted at the commonly used conditions or according to the suppliers' suggestion.

### Example 1: Gene Cloning and Construction of Expression Vectors

The amino acid sequence of wild-type lysine decarboxylase derived from Plesiomonas shigelloides, which can be retrieved from NCBI, and the corresponding nucleic acid was then synthesized by a vendor using conventional techniques in the art and cloned into the expression vector pACYC-Duet-1. The recombinant expression plasmid was transformed into E. coli BL21 (DE3) competent cells under the conditions of 42° C. and thermal shock for 90 seconds. The transformation solution was plated on LB agar plates containing chloramphenicol, which was then incubated overnight at 37° C. to obtain recombinant transformants.

### Example 2: Expression of Lysine Decarboxylase Polypeptides

Recombinant E. coli BL21 (DE3) obtained in Example 1 was inoculated into 50 mL of LB medium containing chloramphenicol (peptone 10 g/L, yeast extract powder 5 g/L, sodium chloride 10 g/L, pH 7.0±0.2, 25° C.) in a 250 mL Erlenmeyer flask, which was then cultured in a shaking incubator at 30° C., 250 rpm overnight. When the OD₆₀₀ of the overnight culture reached 2, it was subcultured at the inoculum of 5% (v/v) into a 1000 mL flask containing 250 mL of TB medium (tryptone 12 g/L, yeast extract 24 g/L, disodium hydrogen phosphate 9.4 g/L, dipotassium hydrogen phosphate 2.2 g/L, pH 7.2±0.2, 30° C.), and a final concentration of 6 g/L lactose was added as an inducer, and the culture was placed in a shaking incubator at 30° C., 250 rpm overnight. After induction at 30° C. for 20 h, the culture broth was centrifuged, the cells were harvested, and the supernatant was discarded by centrifugation to obtain the wet cells, and the wet cells obtained was placed in a refrigerator at −20° C. for future usage. If enzyme solution was required, the resulting wet cells were resuspended with 50 mL of PBS buffer (pH 7.4), and the solution was sonicated in an ice bath for 5 min (5 s/s, 50% power). The resulting cell lysate was clarified by centrifugation at 8000 rpm for 10 min at 4° C. using a Thermo Multifuge X3R centrifuge. The clarified supernatant was frozen at −20° C.

### Example 3: Construction of Lysine Decarboxylase Mutant Libraries

All the reagents used here are commercial reagents, preferably the Quikchange kit (supplier: Agilent) was used. The sequence design of the mutagenesis primers was performed according to the instructions of the kit. The PCR reaction consisted of 10 μL of 5×Buffer, 1 μL of 10 mM dNTP, 1 μL of plasmid DNA template (50 ng/μL), 0.75 μL (10 μM) each of the upstream and downstream primers, 0.5 μL of high fidelity enzyme and 36 μL of ddH₂O, The PCR primer has a NNK codon at the mutation position.

PCR amplification steps: (1) 98° C. pre-denaturation for 3 min; (2) 98° C. denaturation for 10 s; (3) annealing and extension for 3 min at 72° C.; (4) repeating step (2) to (3) for 25 times; (5) extension for 10 min at 72° C.; cooling to 4° C., 2 μL of Dpnl was added to the PCR product and the plasmid template was eliminated by overnight digestion at 37° C. The digested PCR product was transformed into E. coli BL21 (DE3) competent cells and plated on LB agar plates containing chloramphenicol to obtain mutagenesis libraries.

### Example 4: High-Throughput Screening of Lysine Decarboxylase Mutagenesis Libraries

For the expression of lysine decarboxylase mutant libraries, the protocol for shake flask preparation was scaled down to 96-well plates. Mutant colonies were picked from the LB agar plates, inoculated into LB medium containing chloramphenicol in a 96-well shallow plate with 200 μL/well LB medium, and it was cultured overnight (18 to 20 hours) at 180 rpm, 80% humidity, 30° C. in a shaking incubator. When the OD₆₀₀ of this overnight culture reached about 2.0, 20 μL was used to inoculate a 96-well deep-well plate with 400 μL/well of TB medium (containing chloramphenicol). The deep-well plate was placed in a shaking incubator at 250 rpm, 30° C., and 80% humidity to continue cultivation. When OD₆₀₀ of deep-well culture reached 0.6˜0.8, IPTG was added at a final concentration of 1 mM as an inducer to induce expression, and the expression undertook at 30° C. overnight. Finally, the deep-well plate was taken out and centrifuged at 4000 rpm for 10 min; the culture medium was removed, and the pellets of wet cells in the deep-well plates were placed in the freezer at −20° C.

The assay protocol for screening the mutant library targeting the catalytic conversion of L-lysine to 1.5-diaminopentane was as follows:

To the 96-well plate containing wet cells, 300 μL/well of lysis solution (containing 0.2 mM PLP, 0.1 M phosphate buffer pH 6.0) was added by a dispenser, sealed with the membrane and put on a plate oscillator at 700 rpm for 1 h. 500 g/L L-lysine hydrochloride solution (containing 0.2 mM PLP) was prepared, and it was added into deep-well plates by 180 μL/well to make the final concentration of L-lysine hydrochloride 450 g/L. 20 μL/well of the cell lysate added into the 96 deep-well plates, and the total volume of the reaction was 200 μL. The 96-well plate was heat-sealed with an aluminum film, and put in the shaking incubator at 35° C., 200 rpm for 20 h. At the end of the reaction, 20 μL of the reaction solution was taken and quenched with 280 μL acetonitrile, shaking at 700 rpm for 1 h. The quenched reaction was centrifuged, and the supernatant was diluted for HPLC analysis to detect the conversion.

### Example 5: HPLC Analysis Method

High-throughput screening analytical method: analytical column Chirex 3126(D)-penicillamine 30*4.6 mm, mobile phase 1 mM copper sulfate; flow rate: 1.2 mL/min; column temperature: 45° C.; detection wavelength: 280 nm; injection volume: 10 μL.

### Example 6: Fermentation and Downstream Processing

A single microbial colony of E. coli BL21 (DE3) containing a plasmid bearing the target lysine decarboxylase gene was inoculated into a 50 mL of LB broth containing 30 μg/mL chloramphenicol (5.0 g/L Yeast Extract, 10 g/L Tryptone, 10 g/L NaCl). Cells were incubated overnight for 16 hours with shaking at 250 rpm in a 30° C. shaker. When the OD₆₀₀ of the culture reached 3.5 to 4.5, the culture was used to inoculate medium in fermentor immediately.

A 1.0 L fermenter containing 0.4 L of growth medium was sterilized in a 121° C. autoclave for 30 minutes. The fermentor was inoculated with the abovementioned culture. Temperature of fermentor was maintained at 37° C. The growth medium in fermentor was agitated at 200-1000 rpm and air was supplied to the fermentation vessel at 0.4-0.8 L/min to maintain a dissolved oxygen level no less than 60%. The culture was maintained at pH 6.5-7.0 by addition of 25-28% v/v ammonium hydroxide. Cell growth was maintained by feeding a feed solution containing 500 g/L of dextrose monohydrate, 12 g/L ammonium chloride, and 5 g/L magnesium sulfate heptahydrate. After the OD₆₀₀ of culture reached 25±5, the temperature of fermentor was decreased and maintained at 30° C., and the expression of target polypeptides was induced by the addition of α-lactose to a final concentration of 15 g/L. Fermentation process then continued for additional 16-22 hours. After the fermentation process was complete, cells were harvested using a Thermo Multifuge X3R centrifuge at 8000 rpm for 10 minutes at 4° C. Harvested cells were used directly in the downstream recovery process or stored frozen at −20° C.

6 g of cell pellet was resuspended in 30 mL of 100 mM potassium phosphate buffer containing 250 μM pyridoxal 5′-phosphate (PLP), pH 7.5 at 4° C. The cells were then homogenized twice into cell lysate using a homogenizer at 800 bar to release Lysine decarboxylase. The cell lysate was clarified using a Thermo Multifuge X3R centrifuge at 8000 rpm for 10 minutes at 4° C. The clarified supernatant was frozen at −20° C.

### Example 7: Reaction Process for the Catalytic Generation of 1.5-Diaminopentane by Engineered Lysine Decarboxylase Polypeptides

This embodiment provides a method for the enzymatic preparation of 1,5-diaminopentane comprising the following steps:

S1, 50 g of L-lysine hydrochloride was dissolved in 60 mL of water to obtain the substrate solution and the substrate solution was placed in a reaction flask.

S2: 0.5 g of wet cells with the engineered lysine decarboxylase as made in Example 6 was added to a 2 mL centrifuge tube, and 1.5 mL of pure water was added to thoroughly resuspend the wet cells. The resuspended cells were added to the reaction flask, and the centrifuge tube was rinsed with 1 mL of water which was added to the reaction flask as well. Then, 2 mL of 10 mM pyridoxal phosphate solution was added into the reaction flask to start the decarboxylation reaction, and the temperature of the decarboxylation reaction was controlled at 30° C. The reaction was stirred at a speed of 300 rpm for 6 hours, and the conversion rate was detected by the analytical method mentioned in Example 5 and shown in the table below.

S3: 100 mL of methanol was added to the above reaction solution, and it was heated to inactivate the decarboxylase. The quenched reaction was cooled at room temperature, and then its pH was adjusted to 2 using hydrochloric acid; filtration was carried out to obtain the filtrate; then the filtrate was subject to filtration under reduced pressure until crystals precipitated out of the liquid surface. The filtration was stopped, and the liquid was cooled at room temperature. Then, 250 mL of ethanol was added, shaken, and it was kept at 4° C. overnight. Finally, filtration was carried out to remove the ethanol and water; and then the solids were rinsed with 100 mL of ethanol, and dried in an oven at 50° C. for 12 h to obtain 1,5-diaminopentane hydrochloride. To obtain 1,5-diaminopentane, aqueous sodium hydroxide was added, and 1,5-diaminopentane was isolated by distillation.

| SEQ ID No | Conversion Rate |
|---|---|
| 40 | 97.59% |
| 62 | 98.35% |
| 98 | 99.95% |
| 152 | >99.99% |
| 170 | >99.99% |

### Example 8: Reaction Process at Elevated Scale

S1: 5 g of wet cells with the engineered lysine decarboxylase SEQ ID No: 170 as produced in Example 6 above was put into 630 mL of pure water, 0.055 g of pyridoxal phosphate was weighed and added into the system, and the temperature of the reaction was controlled to be 35° C. or 40° C., stirred at 300 rpm, and 500 g of L-lysine hydrochloride was slowly added into the system, and the reaction was carried out for 6 h to obtain the reaction solution, which was detected by the analytical method mentioned in Example 5, and the conversion rate exceeded 99.99%.

S2: The pH of the above reaction solution was adjusted to 1 with hydrochloric acid, filtration was carried out to obtain the filtrate, and the filtrate was electrodialyzed. The aqueous solution of 1,5-diaminopentane obtained by electrodialysis was distilled to obtain 1,5-diaminopentane.

### Example 9: Reaction Process for the Catalytic Generation of 1.5-Diaminopentane by Engineered Lysine Decarboxylase Polypeptide (SEQ ID No: 144)

This embodiment provides a method for the enzymatic preparation of 1,5-diaminopentane at different substrate loadings, which comprises the following steps:

S1, 50 g, 60 g and 65 g of L-lysine hydrochloride were added to 60 mL, 53 mL and 49 mL of water, respectively, to obtain a substrate stock solution, and the substrate solution was added in reaction flasks.

S2: 0.5 g, 0.6 g, 0.65 g of wet cells with the engineered lysine decarboxylase as made in Example 6 was added to a 2 mL centrifuge tube, respectively. 1.5 mL of pure water was added to thoroughly resuspend the wet cells. The resuspended cells were added to the reaction flask, and the centrifuge tube was rinsed with 1 mL of water which was added to the reaction flask as well. Then, 2 mL of 10 mM pyridoxal phosphate solution was added into the reaction flasks to start the decarboxylation reaction and the temperature of the decarboxylation reaction was controlled at 35° C., and the reaction was stirred at a speed of 300 rpm for 6 hours., Then, conversion rate was detected by the analytical method mentioned in Example 5, and the conversion rates were >99.99%, 97.52%, and 96.94%, respectively.

S3, 100 mL of methanol was added to the above reaction solution, the solution was heated to inactivate the decarboxylase. The quenched reaction was cooled at room temperature, and then its pH was adjusted to 2 using hydrochloric acid; filtration was carried out to obtain the filtrate; then filtrate was subject to filtration under reduced pressure until crystals precipitated out of the liquid surface. The filtration was stopped, and the liquid was cooled at room temperature. Then, 250 mL of ethanol was added, shaken, and it was kept at 4° C. overnight. Finally, filtration was carried out to remove the ethanol and water, and then the solids were rinsed with 100 mL of ethanol, and dried in an oven at 50° C. for 12 h to obtain 1,5-diaminopentane hydrochloride. To obtain 1,5-diaminopentane, aqueous sodium hydroxide was added and 1,5-diaminopentane was isolated by distillation.

1. An engineered lysine decarboxylase polypeptide, which is capable of converting L-lysine to 1,5-diaminopentane and carbon dioxide; wherein the polypeptide comprises an amino acid sequence that differs from the sequence of SEQ ID NO: 2 by one or more residues at residue positions corresponding to the following: X5, X11, X12, X13, X24, X48, X85, X95, X99, X108, X111, X116, X119, X143, X144, X145, X316, X334, X368, X378, X383, X422, X440, X441, X445, X516, X521, X551, X561, X591, X595, X693, X694, X701, X710; the polypeptide has lysine decarboxylase activity and an amino acid sequence with at least 99.3% sequence identity to the reference sequence of SEQ ID No: 2.

2. The engineered lysine decarboxylase polypeptide of claim 1, wherein the amino acid sequence of said lysine decarboxylase polypeptide further comprises one or more of the following amino acid residues:
X5 is V;
X11 is A, C, P, R, T or S;
X12 is E, V, R, L, T, Q or K;
X13 is I, W, V, P or Y;
X24 is K;
X48 is S;
X85 is D;
X95 is G;
X99 is D, A, G, Y, T, S or Q;
X108 is Q;
X111 is I;
X116 is Q;
X119 is P, D or R;
X143 is P;
X144 is L, F, Q, R or E;
X145 is D, V, W or L;
X316 is E;
X334 is I;
X368 is C;
X378 is V;
X383 is E;
X422 is Q;
X440 is R;
X441 is Q or R;
X445 is Y, L or A;
X516 is R;
X521 is L or M;
X551 is L, Q or N;
X561 is E;
X591 is G or S;
X595 is R;
X693 is V;
X694 is Q;
X701 is G;
X710 is Q;
wherein the numbering refers to SEQ ID NO:2.

3. The engineered lysine decarboxylase polypeptide of claim 1, wherein said engineered polypeptide comprises (a) an amino acid sequence selected from the group consisting of SEQ ID NOs: 4, 6, 8, 10, 12, 14, 16, 18, 20, 22, 24, 26, 28, 30, 32, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, and 170; or, alternatively, (b) an amino acid sequence having at least 95% sequence identity to one of the reference sequences set forth in part (a) above.

4. The engineered lysine decarboxylase polypeptide of claim 1, wherein said engineered lysine decarboxylase polypeptide is capable of converting L-lysine to 1,5-diaminopentane and carbon dioxide under reaction conditions comprising about 10 g/L-650 g/L L-lysine hydrochloride, about 0.1 mM-1 mM PLP, and a temperature of 10-60° C.

5. A polynucleotide encoding the polypeptide of claim 3.

6. The polynucleotide of claim 5, wherein the polynucleotide sequence is selected from the group consisting of SEQ ID No: 3, 5, 7, 9, 11, 13, 15, 17, 19, 21, 23, 25, 27, 29, 31, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, and 169.

7. An expression vector comprising the polynucleotide of claim 5.

8. A host cell comprising the expression vector of claim 7.

9. A method of preparing a lysine decarboxylase polypeptide, which comprises the steps of culturing the host cell of claim 8 and obtaining a lysine decarboxylase polypeptide from the culture.

10. A lysine decarboxylase catalyst, which comprises the culture obtained in claim 9, cells or culture fluid containing the lysine decarboxylase polypeptide obtained from the culture, or a product processed therefrom; wherein said product is an extract obtained from the cells, an isolated product obtained by isolation or purification of the lysine decarboxylase in the extract, or an isolated product obtained by immobilization of the cells and extracts thereof, or an immobilized product obtained by immobilizing the extracts or isolated products of the extracts.

11. A process of preparing a compound of formula (I): 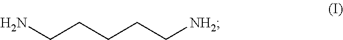
wherein the process comprises the step of contacting L-lysine, a compound of formula (II), with the engineered polypeptide of claim 1 in a suitable solvent under reaction conditions suitable for converting the compound of formula (II) to 1,5-diaminopentane of formula (I);

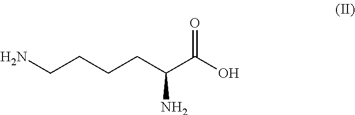

12. The process of claim 11, wherein the reaction conditions comprise a temperature of from 10° C. to 60° C.; 10 g/L-650 g/L L-lysine hydrochloride, about 0.1 mM-1 mM PLP.

13. The process of claim 12, wherein said reaction conditions comprise a temperature of from 30° C. to 40° C.


SEQUENCE LISTING (170 sequences)

SEQ ID NO: 1 - DNA, 2136 bp, synthetic construct
  source 1..2136: mol_type = other DNA
atgaacatcatcgcgatcatgaaccacctgggtgcatttttcaaggaggaaccgattcgcgaactgcatgcaagcctggaagaaaaaggtttccaggtggtgtacccgaatgatcgcgaagatctgctgaaactgctggaaaataacgcccgcgttgctggtgtggtgtttgattgggataagtacagcctggacctgtgtcgtgatattaacgaactgaacgagaagctgccgctgtttgcatttgcaaatacccatagcaccctggatgtgagcctgaatgatctgcgtctgaatgttaccttcttcgagtacgccctggatatggccagcgatattaccctgaaaatcgagcaggcagtggccgcttatgttgatgatattctgccgccgctgaccaaagcactgtttaaatatgtggaggagggtaaatacaccttctgcaccccgggtcacatgggtggtacagcatttcagaaaagcccggttggtagcattttctacgactttttcggtgagaacaccatgaagagcgacattagtatcagcgtgggcgaactgggcagtctgttagatcatagcggcccgcataaagaagcagaagaatatatcgcacgtaccttcaacgccgaacgcagttatatggttaccaacggcaccagcaccgccaataagattgttggcatgtacagcgccccggccggtagcaccatcatgatcgacagaaattgccacaagagcctgacccatctgatgatgatgagtgatgtgaccccgctgtatttccgtccgaccagaaatgcatacggtattctgggtggtatcccgcatagcgaattcactcgcgaaaccattgagcagaagatcgcagaaaccccgaatgccgaatggccggttcatgctgttgttaccaatagcacctacgacggtctgctgtataacaccgataccattaagaagaccctggacgtgaagagtatccatttcgatagcgcatgggtgccgtataccaattttcatccgatctacaagggtaaatgcggcatgagtggcgatcgcgttccgggtaaagttttctatgagacccagagcacccacaaactgctggctgcattttctcaggcaagcatgattcacgtgaagggcgatattaacgaggagacctttagcgaagcctacatgatgcataccagcaccagtccgcattatggcattgtggcaagcaccgaaaccgcagcagccatgatgaaaggcaatgccggtaaacgtctgatccagggtagcattgatcgtgccattcgctttcgcaaagagattaagaagctgcgtagcgaatgcgatggttggtttttcgatgtgtggcagccggaaaatatcgatgaagtggcatgctggaacctggatccgaaagatggctggcatggttttaaggatatggacgaaaaccacatgttcctggacccgattaaggtgaccctgctgacccctggtctgtcaaaagaaggtaccctggaagaacagggtatcccggcaagtctggtggctaaatttctggatgaccgcggtattatcgtggaaaaaaccggtccgtacaacctgctgtttctgtttagcatcggcatcgataagaccaaggcactgagcctgctgcgcggtttaaccgaattcaaacgctgttacgacctgaacatgaaggtgaaaaacgtgctgccgagtgtgtatgcaagcaatccggaattttacgccgagatgcgcattcaggaactggcccaaggtattcataagctgatctgtcaccacaacctgccggaactgatgtataccgcatttgaagtgctgccgaagctggttatgaccccgcatgcagcatttcagcaggaactgcgcggtaatgtggaagaatgttacctggaagagatggtgggcaaaatcaacgccaatatgatcctgccgtacccgccgggtgtgcctctggttatgcctggtgaaatgattaccgaggagagccgcccggtgttagattttctgcagatgctgtgtgagatcggcgcccattttccgggttttgaaaccgatatccacggtgcataccgtcaggccgatggtagatataccgttaaaatcatcaagcagtaa

SEQ ID NO: 2 - protein, 711 aa, Plesiomonas shigelloides
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 3 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgatcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 4 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMAIDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 5 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcttggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 6 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLLGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 7 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctggatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 8 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLDVTFFEYALDMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 9 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcctgtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 10 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGLYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 11 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctgcagatggcgagcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 12 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALQMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 13 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcagggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 14 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLQGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 15 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagctattgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 16 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSYCDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 17 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcttttataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 18 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGFYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 19 - DNA, 2136 bp, synthetic construct
  source 1..2136: mol_type = other DNA
atgaacattattgcgattatgaaccatctgggtgcgattttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 20 - protein, 711 aa, synthetic construct
MNIIAIMNHLGAIFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 21 - DNA, 2136 bp, synthetic construct
  source 1..2136: mol_type = other DNA
atgaacattattgcgattatgaaccatctgggtgagtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 22 - protein, 711 aa, synthetic construct
MNIIAIMNHLGEFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 23 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 24 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 25 - DNA, 2136 bp, synthetic construct
  source 1..2136: mol_type = other DNA
atgaacattattgtgattatgaaccatctgggtgcgtttttcaaagaggagccgattcgtgagctgcatgcgagcctggaggagaagggcttccaggtggtttacccgaacgaccgtgaggatctgctgaagctgctggaaagtaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 26 - protein, 711 aa, synthetic construct
MNIIVIMNHLGAFFKEEPIRELHASLEEKGFQVVYPNDREDLLKLLESNARVAGVVFDWDKYSLDLCRDINELNEKLPLFAFANTHSTLDVSLNDLRLNVTFFEYALDMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 27 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgggctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggcaaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctggcggtcgttacaccgttaaaatcatcaaacagtaa

SEQ ID NO: 28 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNGLRLNVTFFEYALDMASDITLKIEQAVAAYVDDILPPLTKALFKYVEEGKYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQAGGRYTVKIIKQ

SEQ ID NO: 29 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 30 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 31 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccagggtgcttaccgtcaggctgacggtcgttacaccgttaaaatcatcaaacagtaa

SEQ ID NO: 32 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIQGAYRQADGRYTVKIIKQ

SEQ ID NO: 33 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagcaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 34 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKQLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 35 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaaacgactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgagattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgaactgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 36 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKRLRSECDGWFFDVWQPENIDEVACWNLDPKDGWHGFKDMDENHMFLDPIKVTLLTPGLSKEGTLEEQGIPASLVARFLDDRGIIVEKTGPYNLLFLFSIGIDKTKALSLLRGLTEFKRCYELNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 37 - DNA, 2136 bp, synthetic construct
  source 1..2136: mol_type = other DNA
atgaacattattgcgattatgaaccatctgggtgtgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 38 - protein, 711 aa, synthetic construct
MNIIAIMNHLGVFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 39 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcctgtgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 40 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSLCDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 41 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacgatcacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccgctataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 42 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANDHSTLDVSLNDLRLNVTFFEYALDMASDITLKIEQAVAAYVDDILPPLTKALFKYVEEGRYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 43 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagcaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgaacggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 44 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKQLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLNGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 45 - DNA, 2136 bp, synthetic construct
  source 1..2136: mol_type = other DNA
atgaacattattgcgattatgaaccatctgggtgcgtttttcaaagaggagccgattcgtgagctgcataagagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccaatataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagcaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgaacggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 46 - protein, 711 aa, synthetic construct
MNIIAIMNHLGAFFKEEPIRELHKSLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKQLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLNGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 47 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgcagattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 48 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLQIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 49 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaagcggtggcggcgtatgttgacgatattctgccgccgctgaccaaggcgctgttcaaatacgttgaggaaggcgaatacaccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 50 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGEYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 51 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctggcggttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 52 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLAVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 53 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgcgtgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 54 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSACDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 55 - DNA, 2136 bp, synthetic construct
  source 1..2136: mol_type = other DNA
atgaacattattgcgattatgaaccatctggcggcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 56 - protein, 711 aa, synthetic construct
MNIIAIMNHLAAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 57 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgaactgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 58 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYELNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 59 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattagaaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgagattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 60 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIRKLRSECDGWFFDVWQPENIDEVACWNLDPKDGWHGFKDMDENHMFLDPIKVTLLTPGLSKEGTLEEQGIPASLVARFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 61 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgggtgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 62 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLGVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 63 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgtatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgaacggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 64 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLYVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLNGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 65 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattagacgactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgagattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 66 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIRRLRSECDGWFFDVWQPENIDEVACWNLDPKDGWHGFKDMDENHMFLDPIKVTLLTPGLSKEGTLEEQGIPASLVARFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 67 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaaacgactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgagattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatccaacagtaa

SEQ ID NO: 68 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKRLRSECDGWFFDVWQPENIDEVACWNLDPKDGWHGFKDMDENHMFLDPIKVTLLTPGLSKEGTLEEQGIPASLVARFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIQQ

SEQ ID NO: 69 - DNA, 2136 bp, synthetic construct
  source 1..2136: mol_type = other DNA
atgaacattattgcgattatgaaccatctgggtgcgtttttcaaagaggagccgattcgtgagctgcataagagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgaacggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 70 - protein, 711 aa, synthetic construct
MNIIAIMNHLGAFFKEEPIRELHKSLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLNGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 71 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaaacaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaaattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatccaacagtaa

SEQ ID NO: 72 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKQLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIQQ

SEQ ID NO: 73 - DNA, 2136 bp, synthetic construct
  source 1..2136: mol_type = other DNA
atgaacattattgcgattatgaaccatctgtgcgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 74 - protein, 711 aa, synthetic construct
MNIIAIMNHLCAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 75 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgagattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatccaacagtaa

SEQ ID NO: 76 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVARFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIQQ

SEQ ID NO: 77 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgcgcttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 78 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVARFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 79 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgaaatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 80 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGEINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 81 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaaacgactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgagattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 82 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKRLRSECDGWFFDVWQPENIDEVACWNLDPKDGWHGFKDMDENHMFLDPIKVTLLTPGLSKEGTLEEQGIPASLVARFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 83 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaaaaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgagattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 84 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVARFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 85 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattagaaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaaattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatccaacagtaa

SEQ ID NO: 86 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIRKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIQQ

SEQ ID NO: 87 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatggtgcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 88 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMVHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 89 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtggattccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 90 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWIPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 91 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgaaatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgagattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 92 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGEINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVARFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 93 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattagaaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaaattcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 94 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIRKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 95 - DNA, 2136 bp, synthetic construct
  source 1..2136: mol_type = other DNA
atgaacattattgcgattatgaaccatctgggtgcgtttttcaaagaggagccgattcgtgagctgcataagagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgtacgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 96 - protein, 711 aa, synthetic construct
MNIIAIMNHLGAFFKEEPIRELHKSLEEKGFQVVYPNDREDLLKLLENNARVAGVVFDWDKYSLDLCRDINELNEKLPLFAFANTHSTLDVSLNDLRLYVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 97 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgacggttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 98 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLTVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 99 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtcaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 100 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGQRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 101 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgagtgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 102 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLSVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 103 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattagacaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 104 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIRQLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 105 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgaaaccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattagaaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 106 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTETIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIRKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 107 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaatgtctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 108 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKCLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 109 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgggtggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 110 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAGGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 111 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgagtggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 112 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELASGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 113 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgcaggttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 114 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLQVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 115 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacgtgcacggtgcttaccgtcaggctgacggtcgttacaccgttaaaatcatcaaacagtaa

SEQ ID NO: 116 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDVHGAYRQADGRYTVKIIKQ

SEQ ID NO: 117 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaagcggtggcggcgtatgttgacgatattctgccgccgctgaccaaggcgctgttcaaatacgttgaggaaggccaagacaccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 118 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQDTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 119 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgaaaccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcacaagctgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 120 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTETIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHKLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 121 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccagggtgcttaccgtcaggctgacggtcgttacaccgttaaaatcatcaaacagtaa

SEQ ID NO: 122 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIQGAYRQADGRYTVKIIKQ

SEQ ID NO: 123 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaaccggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 124 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEPAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 125 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccaggtgaccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 126 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQVTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 127 - DNA, 2136 bp, synthetic construct
  source 1..2136: mol_type = other DNA
atgaacattattgcgattatgaaccatctgggtgagtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 128 - protein, 711 aa, synthetic construct
MNIIAIMNHLGEFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 129 - DNA, 2136 bp, synthetic construct
  source 1..2136: mol_type = other DNA
atgaacattattgcgattatgaaccatctgggtcggtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtaggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 130 - protein, 711 aa, synthetic construct
MNIIAIMNHLGRFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 131 - DNA, 2136 bp, synthetic construct
  source 1..2136: mol_type = other DNA
atgaacattattgcgattatgaaccatctgggtgcgtggttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 132 - protein, 711 aa, synthetic construct
MNIIAIMNHLGAWFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 133 - DNA, 2136 bp, synthetic construct
  source 1..2136: mol_type = other DNA
atgaacattattgcgattatgaaccatctgggtctgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 134 - protein, 711 aa, synthetic construct
MNIIAIMNHLGLFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 135 - DNA, 2136 bp, synthetic construct
  source 1..2136: mol_type = other DNA
atgaacattattgcgattatgaaccatctgggtgcggttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 136 - protein, 711 aa, synthetic construct
MNIIAIMNHLGAVFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 137 - DNA, 2136 bp, synthetic construct
  source 1..2136: mol_type = other DNA
atgaacattattgcgattatgaaccatctgccggcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 138 - protein, 711 aa, synthetic construct
MNIIAIMNHLPAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 139 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtggaccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 140 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQWTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 141 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaacctcagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 142 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEPQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 143 - DNA, 2136 bp, synthetic construct
  source 1..2136: mol_type = other DNA
atgaacattattgcgattatgaaccatctgggtgcgcctttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 144 - protein, 711 aa, synthetic construct
MNIIAIMNHLGAPFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 145 - DNA, 2136 bp, synthetic construct
  source 1..2136: mol_type = other DNA
atgaacattattgcgattatgaaccatctgggtacgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 146 - protein, 711 aa, synthetic construct
MNIIAIMNHLGTFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 147 - DNA, 2136 bp, synthetic construct
  source 1..2136: mol_type = other DNA
atgaacattattgcgattatgaaccatctgggtgcgtatttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 148 - protein, 711 aa, synthetic construct
MNIIAIMNHLGAYFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 149 - DNA, 2136 bp, synthetic construct
  source 1..2136: mol_type = other DNA
atgaacattattgcgattatgaaccatctgagggcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 150 - protein, 711 aa, synthetic construct
MNIIAIMNHLRAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 151 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatctgggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 152 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDLGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 153 - DNA, 2136 bp, synthetic construct
  source 1..2136: mol_type = other DNA
atgaacattattgcgattatgaaccatctgggtgcgattttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 154 - protein, 711 aa, synthetic construct
MNIIAIMNHLGAIFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 155 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaagatgcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 156 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEDAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 157 - DNA, 2136 bp, synthetic construct
  source 1..2136: mol_type = other DNA
atgaacattattgcgattatgaaccatctgacggcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 158 - protein, 711 aa, synthetic construct
MNIIAIMNHLTAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 159 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagcttaccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 160 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQLTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 161 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatatgggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 162 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDMGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 163 - DNA, 2136 bp, synthetic construct
  source 1..2136: mol_type = other DNA
atgaacattattgcgattatgaaccatctgggtcagtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 164 - protein, 711 aa, synthetic construct
MNIIAIMNHLGQFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 165 - DNA, 2136 bp, synthetic construct
  source 1..2136: mol_type = other DNA
atgaacattattgcgattatgaaccatctgggtaagtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 166 - protein, 711 aa, synthetic construct
MNIIAIMNHLGKFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 167 - DNA, 2136 bp, synthetic construct
  source 1..2136: mol_type = other DNA
atgaacattattgcgattatgaaccatctgagtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacaggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 168 - protein, 711 aa, synthetic construct
MNIIAIMNHLSAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIEQAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ

SEQ ID NO: 169 - DNA, 2136 bp, synthetic construct
  source 1..2136: mol_type = other DNA
atgaacattattgcgattatgaaccatctgggtgcgtttttcaaagaggagccgattcgtgagctgcatgcgagcctggaggagaagggcttccaggtggtttacccgaacgaccgtgaggatctgctgaagctgctggaaaacaatgcgcgcgtggcgggtgtggtttttgactgggataagtatagcctggacctgtgccgtgatatcaacgagctgaatgaaaaactgccgctgttcgcgtttgcgaacacccacagcaccctggacgtgagcctgaacgatctgcgcctgaatgttaccttctttgagtacgcgctggacatggcgagcgatatcaccctgaaaattgaacgggcggtggcggcgtatgttgacgatattctgccgccgctgaccaaggcgctgttcaaatacgttgaggaaggccagtataccttttgcaccccgggtcacatgggtggcaccgcgttccagaagagcccggtgggtagcatcttttacgacttctttggcgagaacaccatgaagagcgatatcagcattagcgttggtgaactgggcagcctgctggaccacagcggtccgcataaagaggcggaggaatacattgcgcgtaccttcaatgcggaacgcagctatatggtgaccaacggtaccagcaccgcgaataagatcgttggcatgtatagcgcgccggcgggtagcaccatcatgattgaccgtaactgccacaaaagcctgacccatctgatgatgatgagcgatgtgaccccgctgtacttccgtccgacccgcaatgcgtatggcatcctgggtggcattccgcacagcgagtttacccgcgaaaccatcgaacagaagattgcggaaaccccgaacgcggaatggccggttcatgcggtggttaccaacagcacctacgacggtctgctgtataataccgataccattaagaaaaccctggacgtgaaaagcatccacttcgatagcgcgtgggttccgtacaccaactttcatccgatttataagggtaaatgcggcatgagcggtgaccgtgtgccgggcaaggttttctacgaaacccagagcacccacaaactgctggcggcgtttagccaagcgagcatgattcatgtgaagggcgatatcaacgaggaaaccttcagcgaggcgtacatgatgcacaccagcaccagcccgcattatggtatcgttgcgagcaccgaaaccgcggcggcgatgatgaagggcaatgcgggtaaacgtctgatccagggcagcattgaccgcgcgatccgttttcgcaaagagattaagaaactgcgtagcgaatgcgacggttggttctttgatgtgtggcaaccggagaacatcgacgaagttgcgtgctggaatctggacccgaaggatggctggcacggtttcaaagacatggatgagaaccatatgtttctggatccgattaaggtgaccctgctgaccccgggcctgagcaaagaaggtaccctggaggaacaaggtatcccggcgagcctggtggcgaagttcctggacgatcgtggcatcattgttgagaaaaccggtccgtacaacctgctgttcctgtttagcatcggcattgacaagaccaaagcgctgagcctgctgcgtggtctgaccgaattcaagcgctgctatgatctgaacatgaaggtgaaaaatgttctgccgagcgtgtacgcgagcaacccggagttttatgcggaaatgcgtatccaggagctggcgcaaggcattcaccgactgatctgccaccataatctgccggagctgatgtacaccgcgttcgaagtgctgccgaaactggttatgaccccgcatgcggcgtttcagcaagaactgcgtggtaacgttgaggaatgctacctggaagagatggtgggcaagattaacgcgaacatgattctgccgtatccgccgggtgttccgctggttatgccgggtgaaatgatcaccgaagaatctcgtccggttctggacttcctgcagatgctgtgcgaaatcggtgctcacttcccgggtttcgaaaccgacatccacggtgcttaccgtcaggctgacggtcgttacaccgttaaaatcatcaaacagtaa

SEQ ID NO: 170 - protein, 711 aa, synthetic construct
MNIIAIMNHLGAFFKEEPIRELHASLEEKGFQVVYPNDREDLLKLLENNARVAGVVFDWDKYSLDLCRDINELNEKLPLFAFANTHSTLDVSLNDLRLNVTFFEYALDMASDITLKIERAVAAYVDDILPPLTKALFKYVEEGQYTFCTPGHMGGTAFQKSPVGSIFYDFFGENTMKSDISISVGELGSLLDHSGPHKEAEEYIARTFNAERSYMVTNGTSTANKIVGMYSAPAGSTIMIDRNCHKSLTHLMMMSDVTPLYFRPTRNAYGILGGIPHSEFTRETIEQKIAETPNAEWPVHAVVTNSTYDGLLYNTDTIKKTLDVKSIHFDSAWVPYTNFHPIYKGKCGMSGDRVPGKVFYETQSTHKLLAAFSQASMIHVKGDINEETFSEAYMMHTSTSPHYGIVASTETAAAMMKGNAGKRLIQGSIDRAIRFRKEIKKLRSECDGWFFDVWQPENIDEVACWNLDPKDGWHGFKDMDENHMFLDPIKVTLLTPGLSKEGTLEEQGIPASLVAKFLDDRGIIVEKTGPYNLLFLFSIGIDKTKALSLLRGLTEFKRCYDLNMKVKNVLPSVYASNPEFYAEMRIQELAQGIHRLICHHNLPELMYTAFEVLPKLVMTPHAAFQQELRGNVEECYLEEMVGKINANMILPYPPGVPLVMPGEMITEESRPVLDFLQMLCEIGAHFPGFETDIHGAYRQADGRYTVKIIKQ